(19) United States
(12) Patent Application Publication
(10) Pub. No.: US 2023/0063965 A1
(43) Pub. Date: Mar. 2, 2023

(54) ARTICLES OF MANUFACTURE COMPRISING ANTI PD-L1 ANTIBODIES AND THEIR USE IN THERAPY

(71) Applicant: Yeda Research and Development Co. Ltd., Rehovot (IL)

(72) Inventors: Rony DAHAN, Rehovot (IL); Noy COHEN SABAN, Rehovot (IL)

(73) Assignee: Yeda Research and Development Co. Ltd., Rehovot (IL)

(21) Appl. No.: 17/794,268

(22) PCT Filed: Jan. 28, 2021
(86) PCT No.: PCT/IL2021/050100

§ 371 Date: Jul. 21, 2022

(30) Foreign Application Priority Data
Jan. 30, 2020 (IL) 272389

(51) Int. Cl. C07K 16/28; A61P 35/00

(52) U.S. Cl. CPC C07K 16/2818; C07K 16/283; C07K 16/2827; A61P 35/00; A61K 2039/507

(57) ABSTRACT

An article of manufacture is provided. The article of manufacture comprising a first binding moiety which specifically binds a human FcgamaRIIB and blocks interaction with IgG antibodies and a second binding moiety which specifically binds a human PD-L1, wherein the second binding moiety which specifically binds the human PD-L1 is an antibody of a human IgG1 isotype.

### RELATED APPLICATION

This application claims priority from Israeli Patent Application No. 272389 filed 30 Jan. 2020, which is incorporated herein by reference in its entirety.

### SEQUENCE LISTING STATEMENT

The ASCII file, entitled 85624 Sequence Listing.txt, created on 25 Jan. 2021, comprising 86,016 bytes, submitted concurrently with the filing of this application is incorporated herein by reference.

### FIELD AND BACKGROUND OF THE INVENTION

The present invention, in some embodiments thereof, relates to articles of manufacture comprising anti PD-L1 antibodies and their use in therapy.

Anti-PD-1/PD-L1 monoclonal antibodies (mAbs) are the paradigm checkpoint inhibitory antibodies, as they demonstrate the most promising clinical activity to-date while eliciting durable responses in treated patients across multiple tumor types. Nevertheless, these mAbs still exhibit notable limitations. Not all cancer types are suitable for this type of therapy, in fact only a portion of patients respond to the treatment. Therefore, effective treatment with anti-PD-1/L1 mAb therapy remains an unmet clinical need. The present inventors previously identified surprising differences in the activity of FcγR pathways that lead to distinct in-vivo mechanisms induced by anti-PD-1 vs. -PD-L1 (Dahan et al. FcγRs modulate the anti-tumor activity of antibodies targeting the PD-1/PD-L1 Axis. Cancer Cell. 14; 28(3):285-95. DOI: 10.1016/j.ccell.2015.08.004).

Mouse anti-PD-L1 antibodies showed augmented anti-tumor effect when an activating-FcγR-binding activity was introduced into the molecules, through an as-yet unclear mechanism that involves modulation of the myeloid cells within the TME. This FcγR-dependent pathway 30 synergizes with the FcγR-independent PD-1/L1 blocking activity of anti-PD-L1 antibodies, thereby augmenting their therapeutic efficacy (Dahan et al. Supra).

However, it is not known whether a similar mechanism is engaged, or can be potentiated, by human anti-PD-L1 drugs.

Three anti-PD-L1 mAbs, Avelumab, Atezolizumab, and Durvalumab, have been approved by the FDA for treatment of different types of lung cancer and urothelial and Merkel cell carcinomas, and their efficacy for additional indications is currently being evaluated in additional clinical trials. Interestingly, while Avelumab is a wild type human IgG1 capable of interacting and inducing various human FcR signaling pathways, both Atezolizumab and Durvalumab were designed to avoid effector functions, and carry IgG scaffolds that are mutated to abolish interactions with FcRs (Akinleye, A., & Rasool, Z. (2019). Immune checkpoint inhibitors of PD-L1 as cancer therapeutics. Journal of Hematology and Oncology, 12(1), 1-13. www(dot)doi(dot)org/10.1186/s13045-019-0779-5).

Thus, these PD-L1 mAbs provide a classic example of the uncertainty in the field regarding how to optimize the drug's scaffold, and whether Fc-recruited effector functions are beneficial or not for the drug's efficacy. Although all three mAbs exhibit clinical activity, no clinical data is available to directly compare their relative efficacy and to determine whether Avelumab benefits from FcR-mediated mechanisms, in addition to its direct Fab-mediated effect on lymphocytes through blocking of the PD-1/PD-L1 axis.

### Additional Background Art

WO2018178122
Chen et al. 2019 Front. Immunol. 10:1-13;
Goletz et al. 2018 Front. Immunol.: 1-12;
Roghanian et al. 2015 Cancer Cell 27(4):473-488;

### SUMMARY OF THE INVENTION

According to an aspect of some embodiments of the present invention there is provided an article of manufacture comprising a first binding moiety which specifically binds a human FcγRIIB and blocks interaction with IgG antibodies and a second binding moiety which specifically binds a human PD-L1, wherein the second binding moiety which specifically binds the human PD-L1 is an antibody of a human IgG1 isotype.

According to an aspect of some embodiments of the present invention there is provided a therapeutically effective amount of each of a first binding moiety which specifically binds a human FcγRIIB and a second binding moiety which specifically binds a human PD-L1, wherein the second binding moiety which specifically binds the human PD-L1 is an antibody of a human IgG1 isotype for use in treating cancer, inflammatory disease or infectious disease.

According to an aspect of some embodiments of the present invention there is provided a method of treating cancer, inflammatory disease or infectious disease in a subject in need thereof, the method comprising administering to the subject a therapeutically effective amount of each of a first binding moiety which specifically binds a human FcγRIIB and a second binding moiety which specifically binds a human PD-L1, wherein the second binding moiety which specifically binds the human PD-L1 is an antibody of a human IgG1 isotype, thereby treating cancer, inflammatory disease or infectious disease.

According to some embodiments of the invention, the first binding moiety and the second binding moiety are in a co-formulation.

According to some embodiments of the invention, the first binding moiety and the second binding moiety are in separate formulations.

According to some embodiments of the invention, the first binding moiety and the second binding moiety compose a multispecific antibody.

According to some embodiments of the invention, the first binding moiety and the second binding moiety compose a bispecific antibody.

According to some embodiments of the invention, the second binding moiety is Avelumab. According to some embodiments of the invention, the first binding moiety which specifically binds the human FcγRIIB is an anti FcγRIIB antibody.

According to some embodiments of the invention, the article, method or use comprises a third binding moiety which binds a cancer antigen.

According to an aspect of some embodiments of the present invention there is provided an anti PD-L1 antibody comprising an Fc region of a human IgG1 isotype having at least 95% identity to SEQ ID NO: 2, the antibody comprising complementary determining regions as set forth in SEQ ID NOs: 18-20 in a heavy chain with an N to C orientation and complementary determining regions as set forth in SEQ ID NOs: 21-23 in a light chain with an N to C orientation, the Fc region comprising at least one mutation and/or modification which specifically enhances binding affinity of the Fc region to human FcγRIIA and/or FcγRIIIA as compared to wild type Fc region of the human IgG1.

According to some embodiments of the invention, the at least one mutation is selected from the group consisting of S238D, S239D, I332E, A330L, S298A, E33A, L334A, G236A and L235V according to EU nomenclature.

According to some embodiments of the invention, the at least one mutation comprises G236A/S239D/A330L/I332E.

According to some embodiments of the invention, the mutation comprise G236A.

According to some embodiments of the invention, the modification is afucosylation.

According to an aspect of some embodiments of the present invention there is provided a method of treating cancer, inflammatory disease or infectious disease in a subject in need thereof, the method comprising administering to the subject a therapeutically effective amount of the antibody of any one of claims 13-17, thereby treating the cancer, inflammatory disease or infectious disease in the subject.

According to an aspect of some embodiments of the present invention there is provided the antibody described herein for use in treating cancer, inflammatory disease or infectious disease.

According to some embodiments of the invention, the method further comprises administering a therapeutically effective amount of a binding moiety which specifically binds a human FcγRIIB and blocks interaction with IgG antibodies.

According to some embodiments of the invention, the method further comprises administering a therapeutically effective amount of a binding moiety which binds a cancer antigen.

According to an aspect of some embodiments of the present invention there is provided the antibody for use with a binding moiety which binds a cancer antigen.

According to some embodiments of the invention, the cancer is a solid tumor cancer.

According to some embodiments of the invention, the cancer is a non-solid tumor cancer.

According to an aspect of some embodiments of the present invention there is provided an isolated polynucleotide comprising a nucleic acid sequence encoding the antibody.

According to an aspect of some embodiments of the present invention there is provided a nucleic acid construct comprising the nucleic acid sequence and a cis-acting regulatory sequence for driving expression of the nucleic acid sequence.

According to an aspect of some embodiments of the present invention there is provided a cell comprising the nucleic acid construct.

According to an aspect of some embodiments of the present invention there is provided a method of producing an antibody, the method comprising:

(a) culturing the cells in a cell culture under conditions which allow expression of the antibody; and
(b) recovering the antibody from the cell culture.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B show that human anti-PD-L1 mAbs bind mouse PD-L1 and block PD-1/PD-L1 interactions in both humans and mice. (FIG. 1A) PD-L1 antigen binding ELISA for two FDA approved human anti-PD-L1 drugs—Avelumab and Atezolizumab. OD450 values were plotted against increasing concentrations of test antibody Avelumab (red) or Atezolizumab (blue) to assess binding to plate-bound mouse or human PD-L1. Avelumab and Atezolizumab cross-react with the mouse PD-L1. (FIG. 1B) PD-1/PD-L1 ELISA blocking assay. OD450 values were plotted against increasing concentrations of test antibody Avelumab (red) or Atezolizumab (blue) in the presence of PD-1 to assess competitive binding to plate-bound mouse or human PD-L1. Avelumab and Atezolizumab were found to block mouse PD-1/PD-L1 interaction in a manner similar to the human interaction.

FIG. 2 shows that Fc-engineering does not impair PD-L1 binding. PD-L1 antigen binding ELISA for the different Fc variants of Avelumab (top) and Atezolizumab (bottom).

OD450 values were plotted against increasing concentrations of test antibodies to assess binding to plate-bound mouse (left) or human (right) PD-L1.

FIG. 3 shows that Abolishing huFc-FcγR engagement from huIgG1 subclass in vivo did not affect anti-tumor response in MC38 tumor model. Anti-tumor response to treatment with Avelumab or Atezolizumab IgG1 or N297A variant. FcvR humanized mice with established MC38 tumors were treated with Avelumab (left) or Atezolizumab (right). IgG1 anti-PD-L1 Abs (blue) did not result in improved antitumor activity compared to N297A variant (red). Data are represented as mean t SEM.

FIGS. 4A-B show that FcγR engagement of Atezolizumab did not alter myeloid cell percentages in the tumor microenvironment (TME). huFcγR mice with established MC38 tumors were treated with the indicated IgG-Fc versions of Atezolizumab. Tumors were harvested and analyzed for the percentages of lymphocytes (A) and myeloid cells (B) in the TME by Flow cytometer. Data are represented as mean t SEM.

FIG. 5 shows that combined targeting of huFcγRIIB and PD-L1 increases the therapeutic effect of Avelumab in MC38 tumor model. huFcγR mice with established MC38 tumors were treated with Avelumab IgG1 in a combinatory treatment with anti-CD32B (FcγRIIB) clone 2B6. One of two independent experiments is shown. Data are represented as mean t SEM.;

FIGS. 6A-G show that Afucosylation of Avelumab Fc glycan results in increased affinity to FcγRIIIA and FcγRIIIB. (FIGS. 6A, 6F) FcγRIIIA binding ELISA for Avelumab IgG1 (blue) and Fc variants: N297A (red) GASDALIE (purple) and Afucosylated (green). (FIGS. 6B-F) FcγR binding ELISA for Avelumab IgG1 (blue) and Fc variants: N297A (red) and Afucosylated (green). GASDALIE and Afucosylated variants have increased affinities to FcγRIIIA. OD450 values were plotted against increasing concentrations of test antibody to assess binding to plate-bound mouse or human PD-L1. (FIG. 6G) Mass spectrometry analysis for percentage of Avelumab Fc glycan fucosylation.

FIG. 7 shows that afucosylation of Avelumab Fc glycan results in increased antitumor effect in vivo in MC38 tumor model. FcγR humanized mice with established MC38 tumors were treated with either Avelumab IgG1 (blue) or Afucosylated Fc variant (green). Afucosylation of Avelumab Fc glycan showed improved antitumor activity compared to Avelumab IgG1 or N297A variant. Data are represented as mean t SEM.

FIGS. 8A-B show that afucosylated Fc variant alters leukocytes percentages in the TME. huFcγR mice with established MC38 tumors were treated with the indicated IgG-Fc versions of Atezolizumab. Tumors were harvest and analyzed for the percentages of lymphocytes (FIG. 8A) and myeloid cells (FIG. 8B) in the TME by Flow cytometer. Data are represented as mean t SEM.

FIG. 9 shows sequences of antibodies of some embodiments of the invention.

### DESCRIPTION OF SPECIFIC EMBODIMENTS OF THE INVENTION

The present invention, in some embodiments thereof, relates to articles of manufacture comprising anti PD-L1 antibodies and their use in therapy.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Anti-PD-1/PD-L1 mAbs are the paradigm checkpoint inhibitory antibodies, as they demonstrate the most promising clinical activity to-date, while eliciting durable responses in treated patients across multiple tumor types. Nevertheless, these mAbs still exhibit notable limitations. Not all cancer types are suitable for therapy, and only a portion of patients respond to the treatment. Therefore, improving anti-PD-1/L1 mAb therapy remains an unmet clinical need.

Whilst reducing the present invention to practice, the present inventors conceived that IgG1 anti PD-L1 antibodies are characterized by compromised efficacy since they have high affinity towards the inhibitory receptor, FcγRIIB, while having a poor affinity to the activating receptors, FcγRIIA and FcγRIIIA. In order to shift this bias, the present inventors engineered the Fc region of Avelumab, a commercial anti PD-L1 IgG1 antibody, to comprise an enhanced affinity towards the activating FcγRs (as compared to the unmodified antibody). Alternatively, the present inventors co-administered the wild type antibody together with an antibody which binds FcγRIIB and competes with binding of IgG1. Both these modalities resulted in enhanced antitumor activity as compared to that of the anti PD-L1 antibody when administered alone, substantiating that immunotherapy targeting PD-L1 can be improved by intervening with FcγRs interactions, which ultimately affects the activity of immune cells in the tumor microenvironment.

Thus, according to an aspect of the invention there is provided an article of manufacture comprising a first binding moiety which specifically binds a human FcγRIIB and blocks interaction with IgG antibodies and a second binding moiety which specifically binds a human PD-L1, wherein the second binding moiety which specifically binds the human PD-L1 is an antibody of a human IgG1 isotype.

Alternatively or additionally, there is provided a therapeutically effective amount of each of a first binding moiety which specifically binds a human FcγRIIB and a second binding moiety which specifically binds a human PD-L1 for use in treating cancer, inflammatory disease or infectious disease, wherein the second binding moiety which specifically binds the human PD-L1 is an antibody of a human IgG1 isotype.

Alternatively or additionally, there is provided a method of treating cancer in a subject in need thereof, the method comprising administering to the subject a therapeutically effective amount of each of a first binding moiety which specifically binds a human FcγRIIB and a second binding moiety which specifically binds a human PD-L1, wherein the second binding moiety which specifically binds the human PD-L1 is an antibody of a human IgG1 isotype, thereby treating cancer, inflammatory disease or infectious disease.

As used herein, a “moiety” refers to an antibody component capable of binding the indicated target.

It will be appreciated that the first binding moiety and the second binding moiety and even at a certain embodiment, the third binding moiety, are distinct (different, i.e., not the same) moieties, although they can be part of a single molecule such as a multispecific binding moiety.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIb, or equivalently Rc-RIIB) receptor. Various properties of human FcγRs are summarized in US20170253659.

According to a specific embodiment, the FcR is human.

The ability to mediate cellular cytotoxic effector functions such as Antibody-dependent cell cytotoxicity (ADCC) and Antibody-dependent cell-mediated phagocytosis (ADCP) is a promising means to enable the enhancement of the antitumor potency of antibodies.

In general, for IgG class antibodies ADCC and ADCP are mediated by engaging of the Fe region with specific so called Fc gamma receptors (FcγRs). There are three classes of receptors in humans: the FcγRI (CD64), FcγRII (CD32) with its isoforms FcγRlla, FcγRllb and FcγRllc, and FcγRIII (CD16) with its isoforms FcγRllla and FcγRlllb. The same region on IgG Fc is bound by all FcγRs, only differing in their affinities with FcγRI having a high affinity and FcγRII and FcγRIII having a low affinity.

ADCC is a mechanism whereby the antibody binds with its Fa, region to a target cell antigen and recruits effector cells by binding of its Fc part to Fc receptors on their surface of these cells, resulting in the release of cytokines such as IFN-7 and cytotoxic granules containing perforin and granzymes that enter the target cell and promote cell death. It was found that in particular the FcγRIIIa plays the most crucial role in mediating ADCC activity to targeted cancer cells.

The majority of innate effector cell types co-express one or more activating FcγR and the inhibitory FcγRIIb, whereas natural killer (NK) cells selectively express one activating Fc receptor (FcγRIII in mice and FcγRIIIA in humans) but not the inhibitory FcγRIIb in mice and humans. Human IgG1 binds to most human Fc receptors and is considered equivalent to murine IgG2a with respect to the types of activating Fc receptors that it binds to.

As used herein “FcγRIIB” refers to the human protein product of the FCGR2B gene.

As used herein “FcγRIIA” refers to the human protein product of the FCGR2A gene.

As used herein “FcγRIIIA” refers to the human protein product of the FCGR3A gene.

The affinity of IgG1 towards the different Fc gamma receptors is provided in Table 2 below and is generally within the μM range 1-5 μM.

According to a specific embodiment, the Fc gamma receptors are human native receptors found in the human body or cells.

Thus, as mentioned the first binding moiety binds human FcγRIIB and blocks (inhibits) binding with the Fc region of an IgG antibody, e.g., IgG1.

According to a specific embodiment, the first binding moiety comprises a variable region which competes with the Fc region of the second binding moiety which binds human PD-L1.

According to a specific embodiment, the first binding moiety comprises a variable region having a higher affinity to FcγRIIB than that of an Fc region of the second binding moiety which binds human PD-L1, in the context of using the anti-PDL1 alone.

As used herein “higher” or “increased” is by at least 10%, 20%, 30%, 40%, 50%, 60%, 70%, 80%, 90% or more say: 100%, 200%, 300% as compared to Fc region of the second binding moiety.

Such an antibody is endowed with a minimum affinity of at least 10⁻⁶ M, 10⁻⁷ M 10⁻⁸ M, 10⁻⁹ M or 10⁻¹⁰ M or even lower when determined by, e.g., surface plasmon resonance (SPR) technology in a BIACORE®. 2000 surface plasmon resonance instrument using the predetermined antigen.

As mentioned, these antibodies compete with binding of the Fc region of the second binding moiety i.e., the anti PD-L1.

Methods of analyzing competition between two proteineceous molecules are well known in the art and include, but are not limited to ELISA, FACS, Western blotting and or other appropriate competitive immunoassays. Such methods often require protein labeling and are well known to those of skills in the art.

As used herein “blocking interaction” means a reduction of at least 10%, 20%, 30%, 40%, 50%, 60%, 70%, 80%, 90% or even complete block in binding of the anti PD-L1 antibody to the FcγRIIB receptor, as determined by an immunoassay, such as described above.

Antibodies which bind FcγRIIB are well known in the art. The Examples section which follows makes use of the 2B6 antibody (SEQ ID NOs: 14 and 15 for the heavy chain and light chain, respectively or SEQ ID NOs: 30 and 31).

Other antibodies which can be used, include, but are not limited to, those provided in US20080044429 and US20080044417, which are hereby incorporated by reference in their entirety.

As used herein “a second binding moiety which specifically binds PD-L1” refers to an antibody component which binds human PD-L1.

As used herein “human PD-L1” refers to the programmed death-ligand 1 (PD-L1) also known as cluster of differentiation 274 (CD274) or B7 homolog 1 (B7-H1), a protein that in humans is encoded by the CD274 gene and refers to an immune checkpoint protein.

PD-L1 is constitutively expressed on immune cells such as T and B cells, dendritic cells (DCs), macrophages, mesenchymal stem cells and bone marrow-derived mast cells (Yamazaki et al., 2002, J. Immunol. 169: 5538-45). According to Keir et al. (2008), Annu. Rev. Immunol. 26: 677-704, PD-L1 can also be expressed on a wide range of non-hematopoietic cells such as cornea, lung, vascular epithelium, liver non-parenchymal cells, mesenchymal stem cells, pancreatic islets, placental synctiotrophoblasts, keratin ocytes, etc. Further, upregulation of PD-L1 is achieved on a number of cell types after activation of said cells. A major role was assigned to PD-L1 in suppressing the immune system during tissue autoimmune disease, allografts, and other disease states.

PD-L1 binds to the programmed death-1 receptor (PD-1) (CD279), which provides an important negative co-stimulatory signal regulating T cell activation. PD-1 can be expressed on all kinds of immune cells such as T cells, B cells, natural killer T cells, activated monocytes and DCs. PD-1 is expressed by activated, but not by unstimulated human CD4⁺ and CD8⁺ T cells, B cells and myeloid cells. Additionally, besides binding to PD-L1, PD-1 also binds to its ligand binding partner PD-L2 (B7-DC, CD273). PD-1 is related to CD28 and CTLA-4, but lacks the membrane proximal cysteine that allows homo-dimerization.

In general, the binding of PD-L1 to PD-1 transmits an inhibitory signal which reduces the proliferation of CD8⁺ T cells.

As used herein “specifically” refers to a binding preference to a target of interest, e.g., PD-L1, FcγRIIB, FcγRIIA as compared to other antigens.

As used herein, the terms “specific binding,” “selective binding,” “selectively binds,” and “specifically binds,” refer to antibody binding to an epitope on a predetermined antigen but not to other antigens. Typically, the antibody (i) binds with an equilibrium dissociation constant (K_{D}) of approximately less than 10⁻⁵ M, such as approximately less than 10⁻⁶ M, 10⁻⁷ M, 10⁻⁸ M, 10⁻⁹ M or 10⁻¹⁰ M or even lower when determined by, e.g., surface plasmon resonance (SPR) technology in a BIACORE®. 2000 surface plasmon resonance instrument using the predetermined antigen, e.g., recombinant DC marker, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen.

Binding of anti PD-L1 antibodies to their target is typically in K_{D} range of 10 nM to 10 μM.

Binding of anti PD-L1 antibodies to FcγRIIB via the Fc region is typically in a K_{D} of about 3 μM.

According to a specific embodiment, the anti-PD-L1 antibodies are human, humanized or chimeric antibodies useful for human therapy.

According to a specific embodiment, the anti-PD-L1 antibodies are of an IgG1 isotype.

According to a specific embodiment, the anti-PD-L1 antibodies comprise an Fc region.

According to a specific embodiment, the anti-PD-L1 is Avelumab (i.e., SEQ ID NO: 2 and 32).

When used in combination with the first binding moiety which specifically binds a human FcγRIIB and blocks interaction with IgG antibodies, the anti-PDL1 is of an IgG1 isotype without a modification in the Fc that affects binding to the FcγRs, e.g., wild type Avelumab.

Alternatively or additionally, the first binding moiety which specifically binds a human FcγRIIB and blocks interaction with IgG antibodies (e.g., 2B6) does not comprise an Fc modification which affects the binding to FcγRs.

According to a specific embodiment, the IgG1 isotype comprises an Fc region comprising at least one mutation and/or modification, which specifically enhances binding affinity of the Fc region to human FcγRIIA and/or FcγRIIIA as compared to wild type Fc region of said human IgG1.

As used herein “enhances” is by at least by at least 10%, 20%, 30%, 40%, 50%, 60%, 70%, 80%, 90% or more say: 100%, 200%, 300% as compared to wild type Fc region of human IgG1.

Thus, the anti PD-L1 antibody may comprise an Fc region of a human IgG1 isotype having at least 95% identity (96%, 97%, 98%, 99% or even 100% identity) to SEQ ID NO: 2, said antibody comprising complementary determining regions as set forth in SEQ ID NOs: 18-20 in a heavy chain with an N to C orientation and complementary determining regions as set forth in SEQ ID NOs: 21-23 (i.e., Avelumab) in a light chain with an N to C orientation, said Fc region comprising at least one mutation and/or modification, which specifically enhances binding affinity of said Fc region to human FcγRIIA and/or FcγRIIIA as compared to wild type Fc region of said human IgG1 (SEQ ID NO:2). Full identity is maintained in the CDR regions.

Examples of such mutations include, but are not limited to S238D, S239D, I332E, A330L, S298A, E33A, L334A, G236A and L235V according to EU nomenclature.

According to other embodiments, the at least one mutation comprises G236A/S239D/A330L/I332E.

According to other embodiments, the at least one mutation comprises G236A.

According to other embodiments, the at least one mutation results in afucosylation.

As used herein “modification” relates to a modified glycosylation on asparagine 297 (N297) according to EU-nomenclature, in the CH₂ domain of the heavy chain.

In general, glycosylated antibodies may comprise two N-linked oligosaccharides at each conserved asparagine 297 (N297), according to EU-nomenclature, in the CH₂ domain. Typically, N-glycans attached to each N297 of the antibody may be of the complex type but also highmannose or hybrid type N-glycans may be linked to each N297 of the antibody. The complex type N-glycosylation may be characterized by a mannosyl-chitobiose core (Man3GlcNAc2-Asn) with variations in the presence/absence of bisecting N-acetylglucosamine and core-fucose, which may be a-1.6-linked to the N-acetylglucosamine that is attached to the antibodies. Furthermore, the complex type N-glycosylation may be characterized by antennary N-acetylglucosamine linked to the mannosyl-chitobiose core (Man3GlcNAc2-Asn) with optional extension of the antenna by galactose and sialic acid moieties. Additionally, antennary fucose and/or N-acetylgalactosamine may be part of the extension of the antenna as well.

According to a specific embodiment, the modification results in fucose-reduced antibody.

The term “fucose-reduced antibody” refers to an antibody comprising 0% to 80% a-1, 6-core fucosylation, also referred to as “afucosylated” or “defucosylated”. In particular, fucose-reduced antibodies of the present invention comprise an Fc region and have two complex N-linked sugar chains bound to the Fc region, wherein among the total complex N-linked sugar chains bound to the Fc region, the content of 1, 6-core-fucose may be from 0% to 80%. The fucose-reduced antibodies of the present invention may contain about 0% to 70%, 0% to 60%, 0% to 50%, 0% to 40%, 0% to 30%, 0% to 20%, 0% to 10% or 10% to 50%, 15% to 50%, 20% to 50%, 25% to 50%, 30% to 50%, 35% to 50%, 40% to 50%, 45% to 50% or 1% to 20%, 1% to 15%, 1% to 10%, 1% to 5% or 5% to 30%, 5% to 20%, 5% to 15% fucosylated N-glycans. The fucose-reduced antibodies of the present invention may preferably contain 0%, 1%, 2%, 3%, 4%, 5%, 6%, 7%, 8%, 9%, 10%, 11%, 12%, 13%, 14%, 15%, 16%, 17%, 18%, 19%, 20.0%, 21%, 22%, 23%, 24%, 25%, 26%, 27%, 28%, 29%, 30%, 40%, 41%, 42%, 43%, 44%, 45.0%, 46%, 47%, 48%, 49%, 50%, 51%, 52%, 53%, 54%, 55%, 56%, 57%, 58%, 59%, 60%, 61.0%, 62%, 63%, 64%, 65%, 66%, 67%, 68%, 69%, 70%, 71%, 72%, 73%, 74%, 75%, 76%, 77%, 78%, 79%, or even 80% fucosylated N-glycans.

Fucose addition or reduction may be catalyzed by alpha-(1.6)-fucosyltransferase (FUT8), which is an enzyme that in humans is encoded by the FUT8 gene.

The production of fucose reduced antibodies can be done in cells in which FUT8 has been silenced (e.g., by genomic modifications or RNA silencing) or by silencing fucose synthesis pathways, silencing additional glycan transferases or by the use of inhibitors as described in the Examples section which follows (e.g., 2-Deoxy-2-fluoro-L-fucose (Carbosynth). Either way such methods are well known in the art and can be found in Biochemistry text books, molecular biology for recombinant expression and in the patent literature, see e.g., U.S. Pat. No. 9,504,702.

The present inventors were able to generate an antibody with fucose content of about 28%.

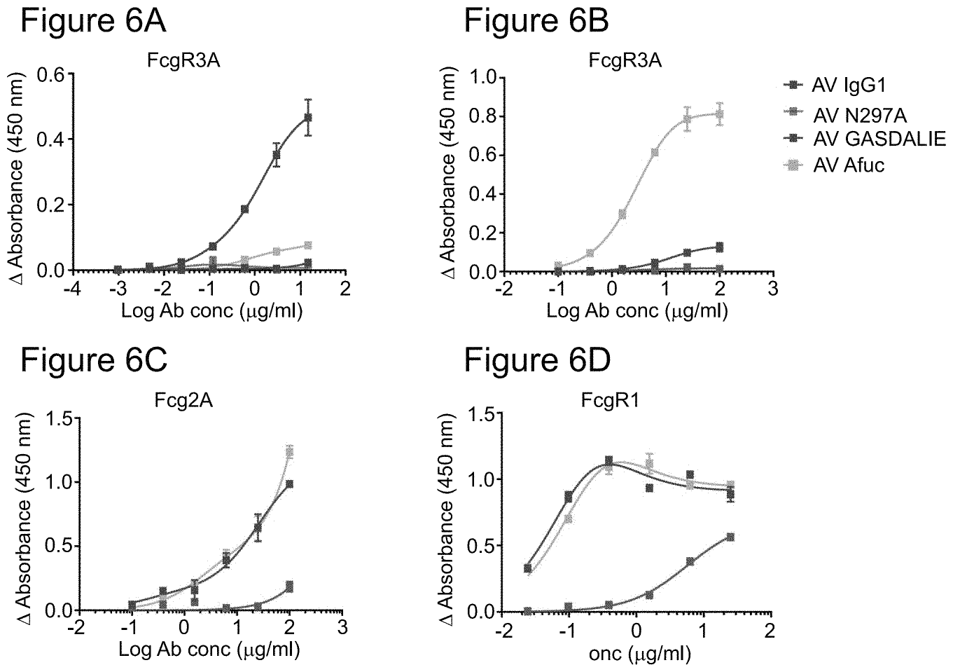

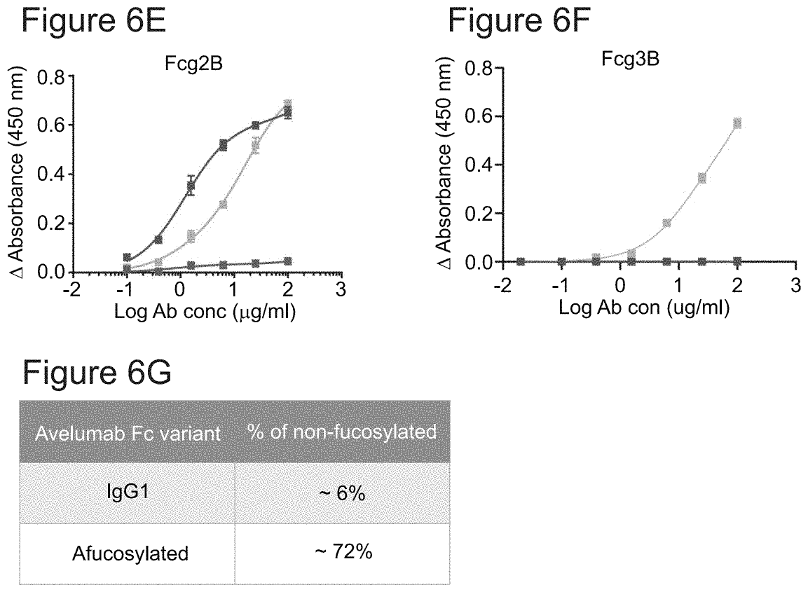

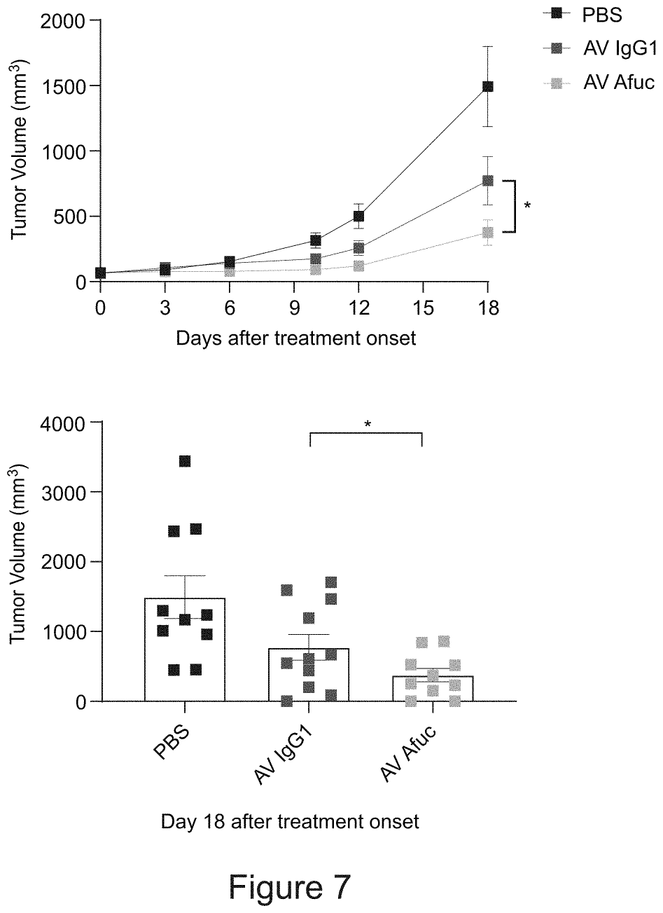

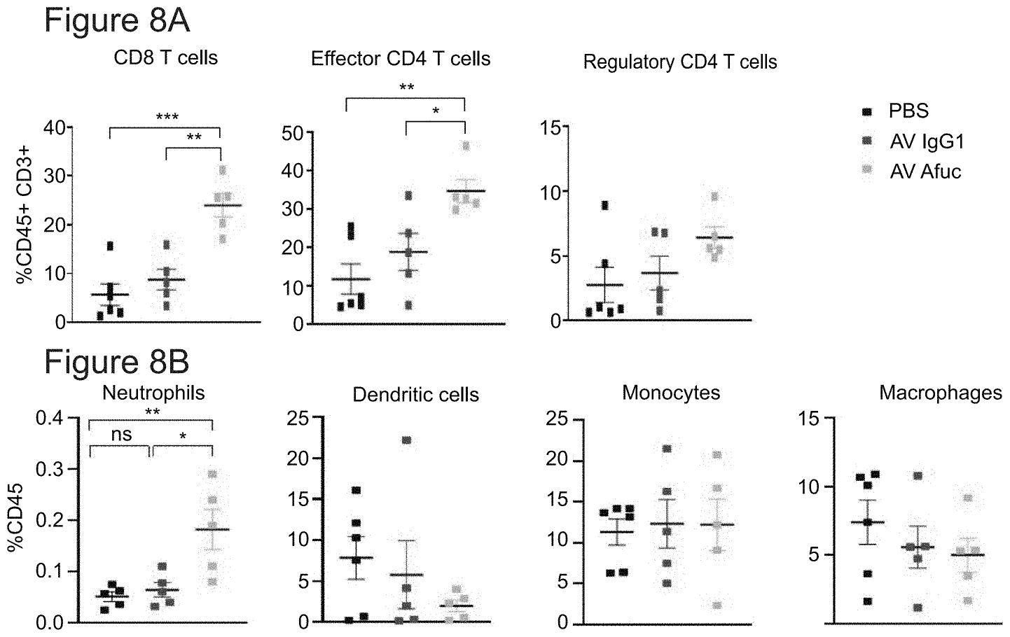

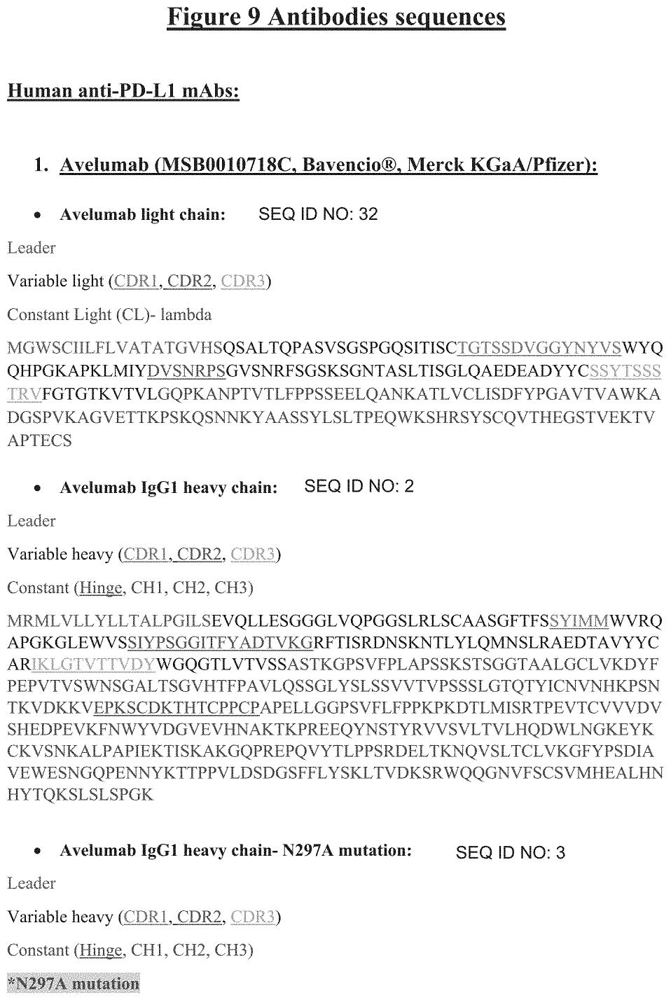

The present inventors showed that the absence of a fucose group from the Fc glycan resulted in specific increased affinity to FcγRIIIA (FIG. 6B) and improved antitumor activity (FIG. 7), as well as altered leukocytes percentages in the TME, giving rise to enrichment in tumor infiltrating lymphocytes as well as neutrophils.

As shown in the Examples section which follows (Table 2) such modifications enhanced binding of the anti PD-L1 antibody (Avelumab) to FcγRIIA and/or FcγRIIIA receptors while ultimately also reduced binding to the inhibitory receptor FcγRIIB (e.g., G236A).

According to a specific embodiment, the light chain of the antibody is that of Avelumab and the heavy chain is as set forth in SEQ ID NO: 2, 4, 5, 6, 7 (Avelumab G236A, GASDLIE, GAALIE, ALIE, respectively).

According to a specific embodiment, the light chain of the antibody is that of Atezolizumab and the heavy chain is as set forth in SEQ ID NO: 8, 10, 11, 12, 13 (Atezolizumab G236A, GASDLIE, GAALIE, ALIE, respectively).

According to a specific embodiment, the antibody is a fucose-reduced antibody.

It will be appreciated, that the article of manufacture can comprise a third binding moiety such as that specifically binding a disease-associated antigen, e.g., cancer antigen.

According to some embodiments, the disease-associated antigen is derived from a protein selected from the group consisting of MART-1/Melan-A, glycoprotein 100 (gp100), tyrosinase, tyrosinase-related protein 1 (TRP1), tyrosinase-related protein 2 (TRP2), BRCA, α-Lactalbumin, HER2/neu, BRAF-V600E, GL261, MUT30, CEA, MUC1, MUC13, CEA, CA 19-9, KRAS, NRAS, RAS, MUC4, prostate cancer antigen (PCA), TRAMP, RANKL, Sperm Protein 17 (SP17), A-kinase anchor protein 4 (AKAP4), Pituitary Tumor Transforming Gene 1 (PTTG1), Aurora kinase A, ovalbumin, bovine serum albumin (BSA), p53, a cancer testis antigen and Adenomatous polyposis coli (APC).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

According to a specific embodiment, the antibody fragments include, but are not limited to, single chain, Fab, Fab′ and F(ab′)₂ fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner. For instance, the anti FcγRIIB be or the anti-cancer antigen can be an antibody fragment, e.g., devoid of an Fc region.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR L1 or L1; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys@, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) disulfide-stabilized Fv (“dsFv”), a genetically engineered antibody including the variable region of the light chain and the variable region of the heavy chain, linked by a genetically engineered disulfide bond.

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);

(vii) Single domain antibodies or nanobodies are composed of a single VH or VL domains which exhibit sufficient affinity to the antigen; and

(viii) Fcab, a fragment of an antibody molecule containing the Fc portion of an antibody developed as an antigen-binding domain by introducing antigen-binding ability into the Fc region of the antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Exemplary methods for generating antibodies employ induction of in-vivo production of antibody molecules, screening of immunoglobulin libraries (Orlandi D. R. et al., 1989. Proc. Natl. Acad. Sci. U.S.A 86:3833-3837; Winter G. et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D. et al., 1985. J. Immunol. Methods 81:31-42; Cote R J. et al., 1983. Proc. Natl. Acad. Sci. U.S.A 80:2026-2030; Cole S P. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumine (BSA)] carriers (see, for example, U.S. Pat. Nos. 5,189,178 and 5,239,078]. Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art.

The antisera obtained can be used directly or monoclonal antibodies may be obtained as described hereinabove.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

As mentioned, the antibody fragment may comprise a Fc region of an antibody termed “Fcab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boemer et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Unless otherwise indicated, an immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., human IgG1, exist in several allotypes, which differ from each other in at most a few amino acids.

According to a specific embodiment, the antibody is of an IgG1 isotype.

As used herein a “multispecific antibody” is an antibody that can bind simultaneously to at least two targets e.g., FcγRIIB and PD-L1.

Specificity indicates how many antigens or epitopes an antibody is able to bind; i.e., bispecific, trispecific, quatraspecific. According to a specific embodiment, the antibody is a bispecific antibody.

Using these definitions, a natural antibody, e.g., an IgG, is bivalent because it has two binding arms but is monospecific because it binds to one epitope.

A “bispecific antibody” is an antibody that can bind simultaneously to two targets which are of different structure, e.g., FcγRIIB and PD-L1.

Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen.

Multispecific, multivalent antibodies are constructs that have more than one binding site of different specificity. For example, a diabody, where one binding site reacts with one antigen and the other with another antigen.

In order to produce the multispecific antibody of some embodiments of the invention, the present moieties can be modified at the Fc region e.g., the CH3 domain (according to kabat) as well known in the art. Such a modification ensures correct assembly of the multispecific antibody via the heavy chains.

Accordingly, the CH3 domain of one heavy chain is altered, so that within the original interface the CH3 domain of one heavy chain that meets the original interface of the CH3 domain of the other heavy chain within the multispecific antibody, an amino acid residue is replaced with an amino acid residue having a larger side chain volume, thereby generating a protuberance within the interface of the CH3 domain of one heavy chain which is positionable in a cavity within the interface of the CH3 domain of the other heavy chain; and the CH3 domain of the other heavy chain is altered, so that within the original interface of the second CH3 domain that meets the original interface of the first CH3 domain within the trivalent, bispecific antibody an amino acid residue is replaced with an amino acid residue having a smaller side chain volume, thereby generating a cavity within the interface of the second CH3 domain within which a protuberance within the interface of the first CH3 domain is positionable (also known as “the knobs-into-holes” approach by Genentech).

According to a specific embodiment, the amino acid residue having a larger side chain volume is selected from the group consisting of arginine (R), phenylalanine (F), tyrosine (Y), tryptophan (W).

According to a specific embodiment, the amino acid residue having a smaller side chain volume is selected from the group consisting of alanine (A), serine (S), threonine (T), valine (V).

According to a specific embodiment, both CH3 domains are further altered by the introduction of cysteine (C) as amino acid in the corresponding positions of each CH3 domain such that a disulfide bridge between both CH3 domains can be formed.

In a specific embodiment, the bispecific comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M., et al., Nature Biotech 16 (1998) 677-681) e.g. by introducing a Y349C mutation into the CH3 domain of the “knobs chain” and a E356C mutation or a S354C mutation into the CH3 domain of the “hole chain”. Thus in a another preferred embodiment, the bispecific antibody comprises Y349C, T366W mutations in one of the two CH3 domains and E356C, T366S, L368A, Y407V mutations in the other of the two CH3 domains or the bispecific antibody comprises Y349C, T366W mutations in one of the two CH3 domains and S354C, T366S, L368A, Y407V mutations in the other of the two CH3 domains (the additional Y349C mutation in one CH3 domain and the additional E356C or S354C mutation in the other CH3 domain forming a interchain disulfide bridge) (numbering always according to EU index of Kabat). But also other knobs-in-holes technologies as described by EP 1 870 459A1, can be used alternatively or additionally. A specific example for the bispecific antibody are R409D; K370E mutations in the CH3 domain of the “knobs chain” and D399K; E357K mutations in the CH3 domain of the “hole chain” (numbering always according to EU index of Kabat).

In another embodiment the bispecific antibody comprises a T366W mutation in the CH3 domain of the “knobs chain” and T366S, L368A, Y407V mutations in the CH3 domain of the “hole chain” and additionally R409D; K370E mutations in the CH3 domain of the “knobs chain” and D399K; E357K mutations in the CH3 domain of the “hole chain”.

In another embodiment the bispecific antibody comprises Y349C, T366W mutations in one of the two CH3 domains and S354C, T366S, L368A, Y407V mutations in the other of the two CH3 domains or the bispecific antibody comprises Y349C, T366W mutations in one of the two CH3 domains and S354C, T366S, L368A, Y407V mutations in the other of the two CH3 domains and additionally R409D; K370E mutations in the CH3 domain of the “knobs chain” and D399K; E357K mutations in the CH3 domain of the “hole chain”.

According to a specific embodiment, Y349C/T366S/L368A/Y407V mutations are introduced to a 1st mAb (e.g., anti FcγRIIB) and S354C/T366W for the 2nd mAb (e.g., anti PD-L1) (Merchant et al., 1998; Ridgway et al., 1996).

Alternatively or additionally, for correct heavy-light chain pairing, at least one of the moieties can be expressed in the CrossMab format (CH1-CL swapping).

The basis of the CrossMab technology is the crossover of antibody domains within one arm of a bispecific IgG antibody enabling correct chain association, whereas correct heterodimerization of the heavy chains can be achieved by the knob-into-hole technology as described above or charge interactions. This can be achieved by exchange of different domains within a Fab-fragment. Either the Fab domains (in the CrossMab^{Fab} format), or only the variable VH-VL domains (CrossMab^{VH-VL} format) or the constant CH1-CL domains (CrossMab^{CH1-CL} format) within the Fab-fragment can be exchanged for this purpose. Indeed, for the CrossMab^{CH1-CL} format the respective original light chain and the novel VL-CH1 light chain do not result in undesired interactions with the respective original and VH-CL containing heavy chains, and no theoretical side products can be formed. In contrast, in the case of the CrossMab^{Fab} format a non-functional monovalent antibody (MoAb) as well as a non-functional Fab-fragment can be formed. These side products can be removed by chromatographic techniques. In the case of the CrossMab^{VH-VL} format an undesired side product with a VL-CH1/VL-CL domain association known from Bence-Jones proteins can occur between the VL-CH1 containing heavy chain and the original unmodified VL-CL light chain. The introduction of repulsive charge pairs based on existing conserved charge pairs in the wildtype antibody framework into the constant CH1 and CL domains of the wildtype non-crossed Fab-fragment can overcome the formation of this Bence-Jones-like side product in the CrossMab^{VH-VL+/-} format. More details on CrossMab Technology can be found in Klein et al. Methods 154, 1 Feb. 2019, Pages 21-31c.

Alternatively, multispecific e.g., bispecific antibodies described herein can be prepared by conjugating the moieties using methods known in the art. For example, each moiety of the multispecific antibody can be generated separately and then conjugated to one another. A variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. (USA) 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

Alternatively or additionally, the conjugation of each moiety of the multispecific antibody can be done via sulfhydryl bonding of the C-terminus hinge regions of the two heavy chains. In a specific embodiment, the hinge region is modified to contain an odd number of sulfhydryl residues, preferably one, prior to conjugation.

According to a specific embodiment, the multispecific antibody is a trispecific antibody, where one moiety binds PD-L1, a second moiety binds FcγRIIB and a third moiety binds a disease associated antigen.

Regardless of the configuration, the binding moiety/antibody can be produced by recombinant DNA technology.

To express recombinant binding moieties in mammalian cells, a polynucleotide sequence encoding the binding moiety is ligated into a nucleic acid construct suitable for cell expression. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors).

A variety of prokaryotic or eukaryotic cells can be used as host-expression systems to express the binding moieties e.g., antibodies, of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the binding moieties e.g., antibodies of some embodiments of the invention.

Examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art and are further described hereinbelow can also be used by some embodiments of the invention.

Recovery of the recombinant binding moieties e.g., antibodies is effected following an appropriate time in culture. The term “recovering” refers to collecting the whole fermentation medium containing the binding moieties e.g., antibodies and need not imply additional steps of separation or purification. Notwithstanding the above, recombinant binding moieties of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

As mentioned, the binding moieties and combinations thereof can be used in disease treatment.

As used herein “disease” refers to any disease or medical condition which can be ameliorated by the inhibition of the PD-1/PD-L1 axis.

According to a specific embodiment, the disease is selected from the group consisting of a cancer disease, an inflammatory disease, a virus infectious disease and an autoimmune disease.

Inflammatory diseases—Include, but are not limited to, chronic inflammatory diseases and acute inflammatory diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like β-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graf Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

According to a specific embodiment, the cancer is a solid tumor cancer.

According to a specific embodiment, the cancer is a non-solid tumor.

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

In particular, cancer disease may be selected from Melanoma, Carcinoma, Lymphoma, Sarcoma, and Mesothelioma including Lung Cancer, Kidney Cancer, Bladder Cancer, Gastrointestinal Cancer, Skin Cancer, Breast Cancer, Ovarian Cancer, Cervical Cancer, and Prostate Cancer. Additionally, inflammatory disease may be selected from Inflammatory Bowel Disease (IBD), Pelvic Inflammatory Disease (PID), Ischemic Stroke (IS), Alzheimer's Disease, Asthma, Pemphigus Vulgaris, Dermatitis/Eczema. Virus infectious disease may be selected from Human Immunodeficiency Virus (HIV), Herpes Simplex Virus (HSV), Epstein Barr Virus (EBV), Influenza Virus, Lymphocytic Choriomeningitis Virus (LCMV), Hepatitis B Virus (HBV), Hepatitis C Virus (HCV). Further, autoimmune disease may be selected from Diabetes Mellitus (DM), Type I, Multiple Sclerosis (MS), Systemic Lupus Erythematosus (SLE), Rheumatoid Arthritis (RA), Vitiligo, Psoriasis and Psoriatic Arthritis, Atopic Dermatitis (AD), Scleroderma, Sarcoidosis, Primary Biliary Cirrhosis, Guillain-Barre Syndrome, Graves' Disease, Celiac Disease, Auto-immune Hepatitis, Ankylosing Spondylitis (AS).

The binding moieties (e.g., antibody/ies) of some embodiments of the invention can be administered to an organism per se, or in a pharmaceutical composition where it is mixed with suitable carriers or excipients.

According to a specific embodiment, the first binding moiety and the second binding moiety are in a co-formulation, i.e., same composition.

According to a specific embodiment, the first binding moiety and the second binding moiety are in separate formulations, i.e., separate compositions that can be administered to the subject concomitantly or sequentially.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the binding moieties (e.g., antibody/ies) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, a pathologic tissue, e.g., cancerous tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide effective levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

It is expected that during the life of a patent maturing from this application many relevant anti PD-L1 antibodies will be developed and the scope of the term anti PD-L1 is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Experimental Procedures

Tumor Challenge and Treatment

Mice 8-10 weeks of age were used in all experiments. MC38 cells (2*10⁶) were implanted subcutaneously (s.c), and tumor volumes were measured every 2-3 days with an electronic caliper and reported as volume using the formula (L₂²*L₁)/2, where L₁ is the longest diameter and L2 is the shortest diameter. Seven days after tumor inoculation, mice were randomized by tumor size (day 0) and received intraperitoneal (i.p) injection of 50-100 μg anti-PD-L1, or control PBS. Mice received an additional 50-100 μg of IgG treatment at days 3 and 6. For the B16 model, mice were challenged with 2*10⁵ B16-F10 cells s.c, and after 3 days (day 0), were treated with 106 irradiated B16-GM-CSF-secreting cells (GVAX) s.c and the first IgG treatment i.p. Additional IgGs were injected at days 3 and 6.

Tissue Processing and Flow Cytometry

For functional experiments, mice were challenged and treated as described above, and were sacrificed at day 8, unless otherwise indicated. Spleens were dissected through a 70 μm nylon cell strainer, incubated with red blood cells lysis buffer (Sigma), and washed. Tumors were mechanically dissected and, in most cases, incubated with DNase (Sigma-Aldrich) and Liberase TL (Roche) before dispersed through a 70 μm nylon cell strainer. Different cell populations were identified after excluding dead cells using live/dead fixable blue dead cell satin kit (Invitrogen). For intracellular staining, cells were fixed and permeabilized with Foxp3 Fix/Perm buffer kit (Bioleagend). CountBright Absolute Counting Beads (Life Technologies) were added prior to acquisition. Cell populations were defined by the following markers: monocytes (CD11b⁺MHC II^{+/-}Ly6C⁺F4/80⁻CD11c⁻), macrophages (CD11b⁺MHCII⁺F4/80⁺CD11c^{+/-}Ly6C⁻Ly6G⁻), immature myeloid cells (CD11b⁺MHCII-F4/80⁺Ly6C⁻Ly6G⁻), neutrophils (CD11b⁺Ly6G⁺Ly6C^{int} MHC II⁻F4/80⁻), dendritic cells (CD11b⁺CD11c⁺MHC II⁺F4/80⁻), CD8 T cells (CD3⁺CD8⁺), CD4 effector T cells (CD3⁺CD4⁺), and CD4 regulatory T cells (CD3⁺CD4⁺Foxp3⁺). Data were acquired on Fortessa flow cytometers (BD) and analyzed using FlowJo software.

ELISA Assays

Binding specificity and affinity of IgG subclasses were determined by ELISA using recombinant PD-L1 (SinoBiological). ELISA plates (Nunc) were coated overnight at 4° C. with recombinant PD-L1 (1 μg/mL/well). All sequential steps were performed at room temperature in 2% BSA. After being washed, the plates were blocked for 1 hr with 1×PBS with 2% Bovine serum Albumin and were subsequently incubated for 1 hr with serially diluted IgGs. After washing, plates were incubated for 1 hr with HRP-conjugated anti-mouse IgG (Jackson ImmunoResearch). Detection was performed by TMB Soluble Reagent (ScyTek Laboratories) and reactions stopped with the addition of 0.18 M sulfuric acid. Absorbance at 450 nm was immediately recorded using a SpectraMax Plus spectrophotometer (Molecular Devices) and background absorbance from negative control samples was subtracted. The following modifications of the protocol described above were performed: (1) For analyzing the ability of anti-PD-L1 antibodies to block PD-1/PD-L1 interactions, ‘competitive ELISA’ was performed. Serial dilutions of anti-PD-L1 drugs were prepared in soluble biotinylated PD-1 (SinoBiological) working solution (1 μg/ml/well) and incubated for 1 hr in room temperature. After washing, plates were incubated for 1 hr with streptavidin-HRP (Jackson ImmunoResearch). (2) For FcγR binding ELISA, human FcγRs soluble ectodomains (2 μg/ml/well) were immobilized to the plate. After being washed, the plates were blocked for 40 minutes with 1×PBS with 10% Bovine serum Albumin and were subsequently incubated for 2 hours with serially diluted IgGs.

Generation and Production of Abs

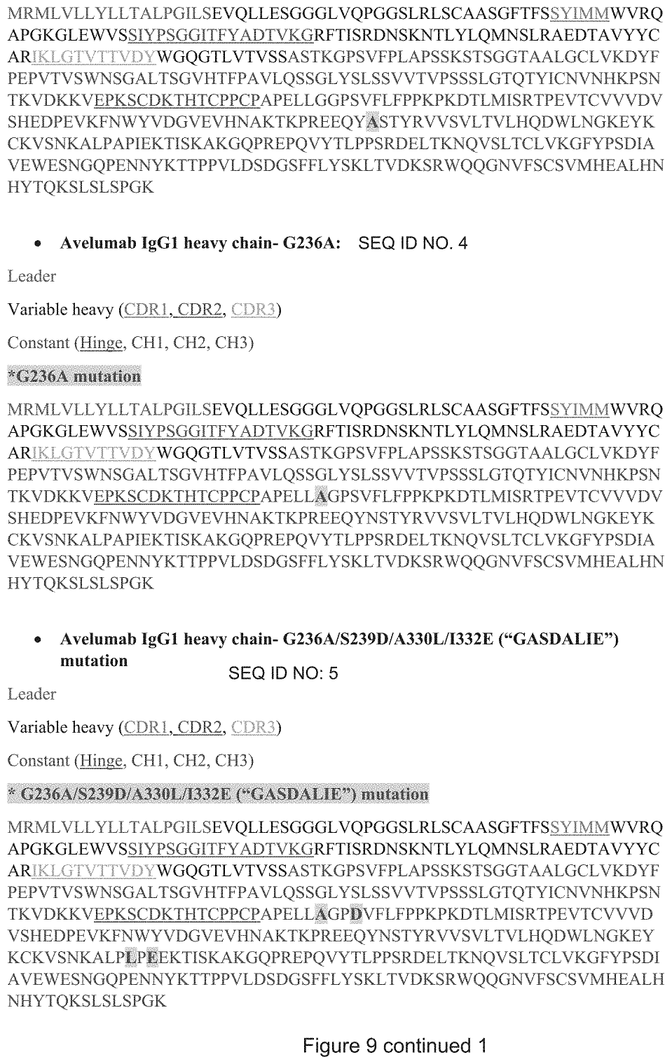

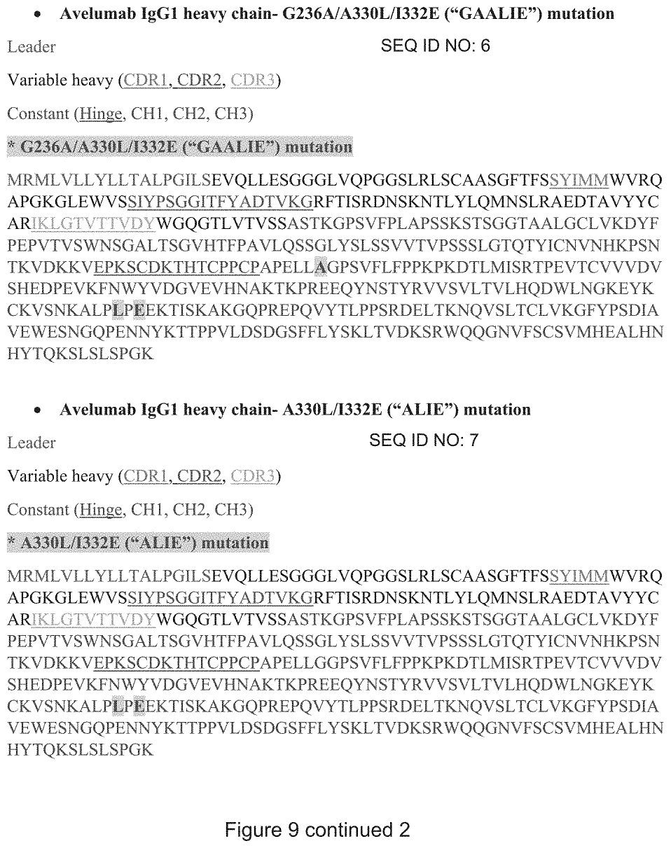

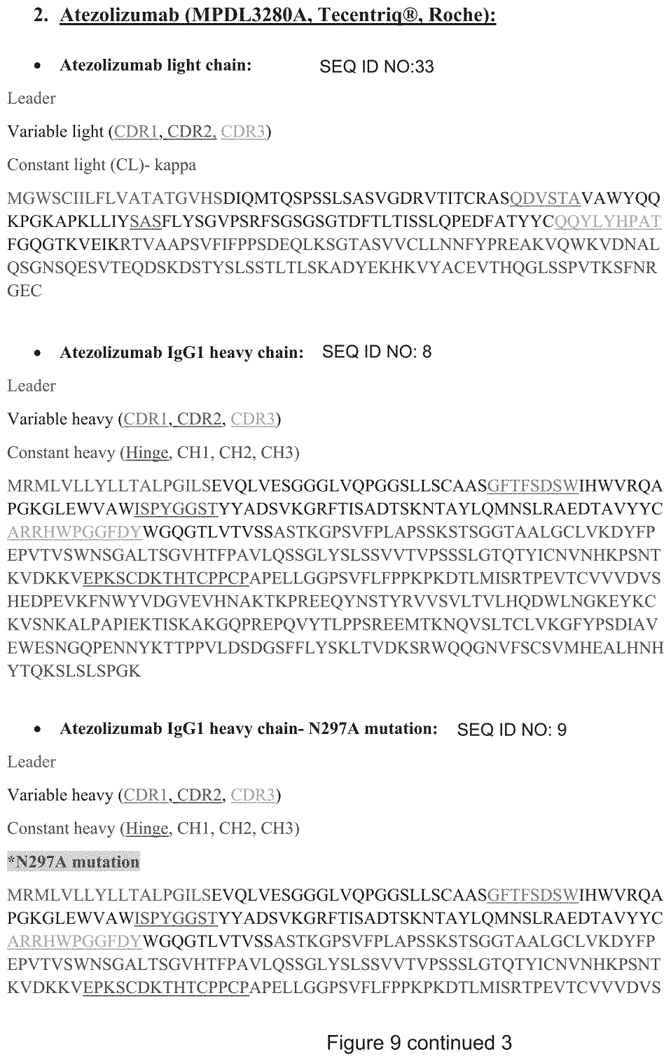

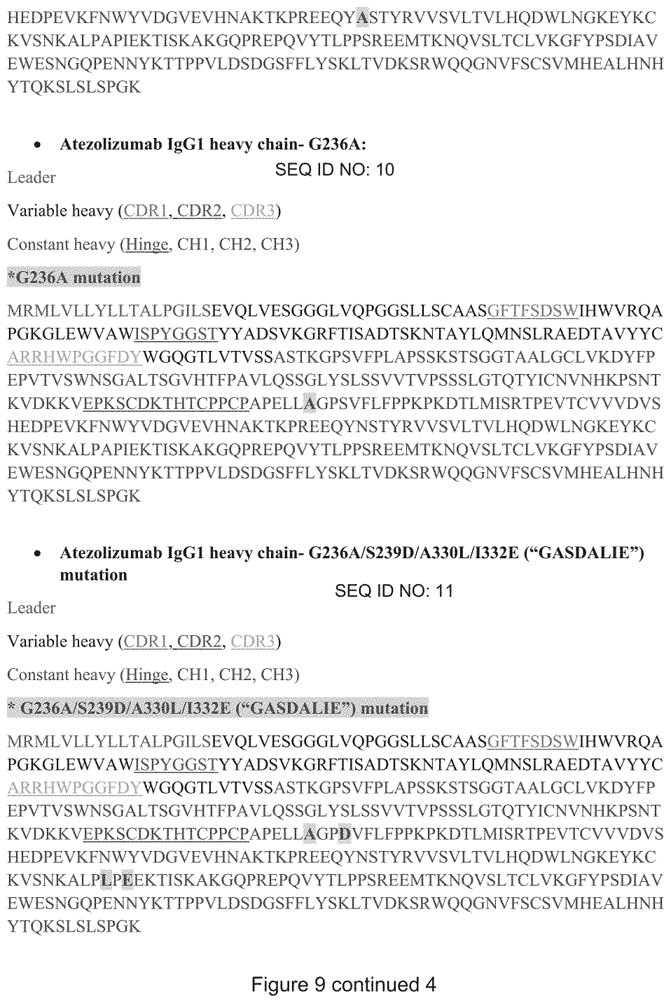

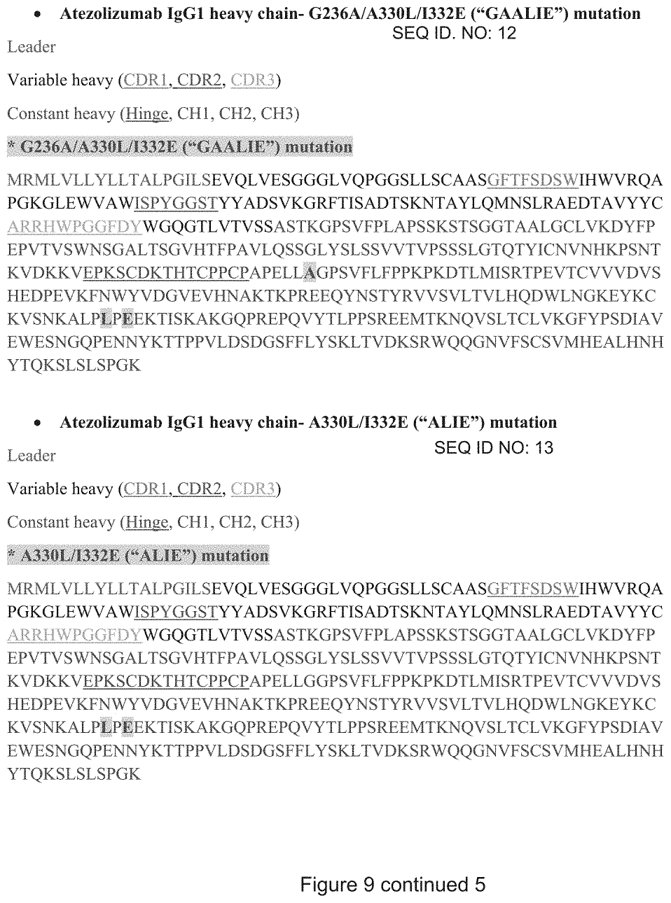

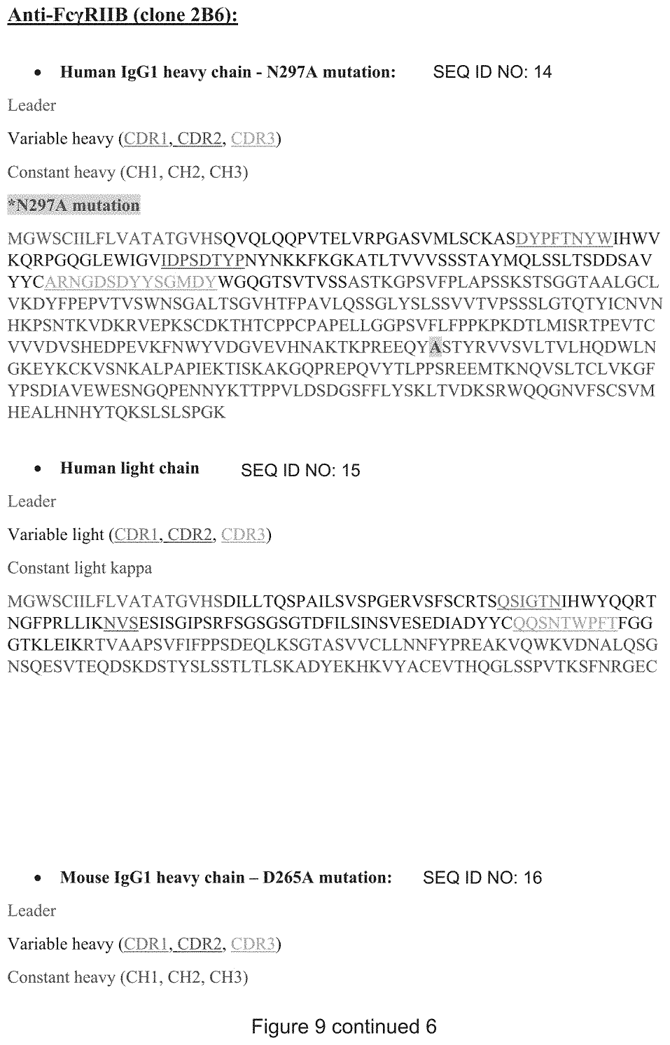

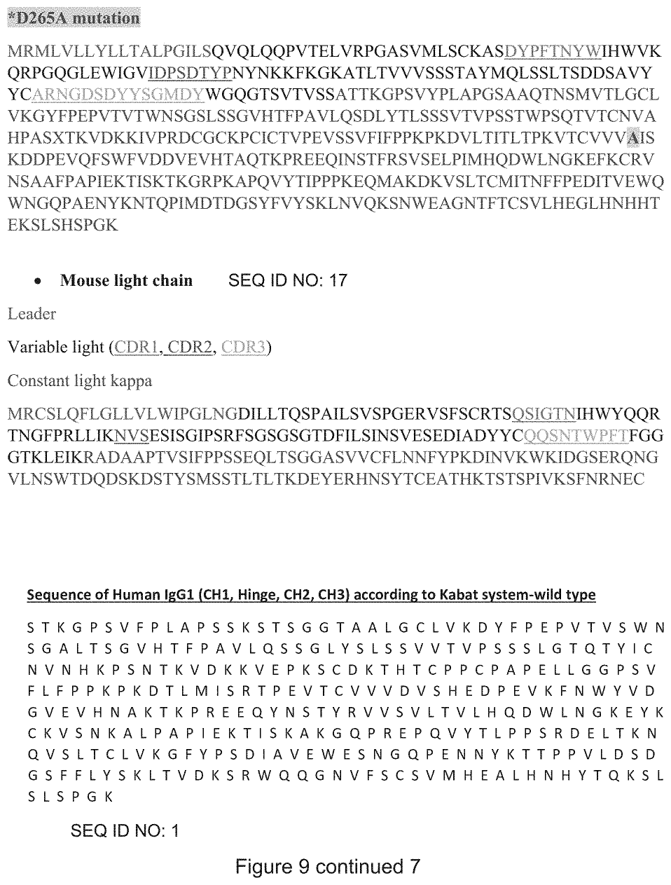

To generate human anti-PD-L1 Abs, Avelumab (Pfizer/Merck KGaA) and Atezolizumab (Roche) sequences (available from the patent applications covering each antibody) were synthesized by BioBasic. Sequences were PCR-amplified and cloned into mammalian expression vectors. Each anti-PD-L1 Ab clone was generated on the background of wild type IgG1, FcγR-binding null IgG1 (N297A), and a panel of Fc variants with defined FcγR-engagement characteristics with the different members of the human FcγR family. Point mutations were introduced in the CH2 domain: “GASDALIE” (G236A/S239D/A330U1I332E), “ALIE” (A330L/I332E), G236A, “GAALIE” (G236A/A330L/I332E) as shown in FIG. 9. For the generation of Fc-domain variants of human IgG1, site-directed mutagenesis using specific primers was performed based on the site-directed mutagenesis by PCR (Agilent Technologies) according to the manufacturer's instructions. Mutated plasmid sequences were validated by direct sequencing (Life science core facility, Weizmann Institute of Science). To produce antibodies, antibody heavy and light chain expression vectors were transfected transiently into Expi293 cells (ThermoFisher). The secreted antibodies in the supernatant were purified by protein G Sepharose 4 Fast Flow (GE Healthcare). Purified antibodies were dialyzed in PBS and sterile filtered (0.22 μm). Purity was assessed by SDS-PAGE and Coomassie staining. For non-fucosylated Fc glycan, 2-Deoxy-2-fluoro-L-fucose (Carbosynth) was added to the transfection medium.

TABLE 1
Summary of FDA approved PD-L1 mAbs.

| Drug | Isotype | Indication |
|---|---|---|
| Avelumab | IgG1 | Merkel cell carcinoma; |
| (Pfizer/Merck KGaA) | | Bladder cancer; RCC |
| Atezolizumab | IgG1-Fc silent | Lung cancer (SCLC and |
| (Genentech-Roche) | | NSCLC); Bladder cancer; |
| | | Breast cancer |
| Durvalumab | IgG1-Fc silent | NSCLC |
| (AstraZeneca) | | |

Mass Spectrometry

Samples were digested with trypsin using the S-trap method, followed by HILIC enrichment of glycopeptides. The resulting peptides were analyzed using nanoflow liquid chromatography (nanoAcquity) coupled to high resolution, high mass accuracy mass spectrometry (Fusion Lumos). The resulting data was searched using Byonic, against the human IgG1 glycosylated peptide and the human Fc glycan database. ID's were verified manually, and quantified using Skyline (v19.1.0.193). FIG. 6G shows the percentages of de-fucosylated forms in the IgG1 and Afucosylated samples as determined by mass-spectrometry analysis).

### Example 2

### Human Anti-PD-L1 mAbs Bind Mouse PD-L1 and Block PD-1/PD-L1 Interactions in Both Humans and Mice

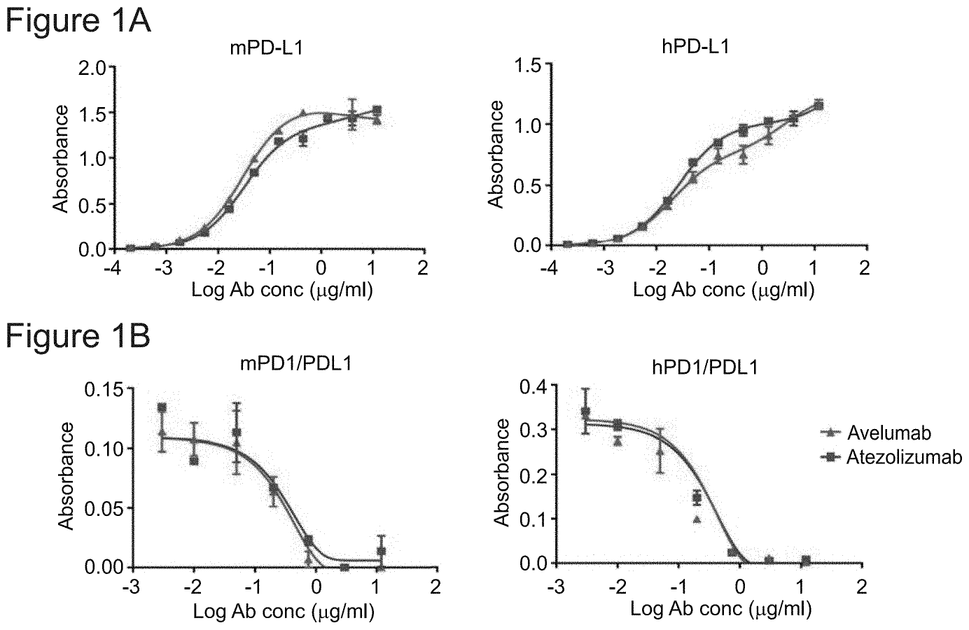

The present inventors aimed at identifying the optimal IgG scaffold of two FDA approved human anti-PD-L1 drugs. The binding and PD-1/PD-L1 blocking activity of the human mAbs were characterized. It was found that both drugs cross-react with the mouse PD-L1 and are able to block mouse PD-1/PD-L1 interaction in a manner similar to their effect on the human PD-1/PD-L1 axis (FIGS. 1A-B). These observations enabled studying the anti-tumor activity of the drugs in the humanized FcγR (huFcγR) mouse strain (Smith P, DiLillo D J, Bournazos S, Li F, Ravetch J V. Mouse model recapitulating human Fc receptor structural and functional diversity. Proc Nal Acad Sci. 2012; 109(16):6181-6186. doi:10.1073/pnas.1203954109).

### Example 3

### Fc-Engineering does not Impair PD-L1 Binding. PD-L1 Antigen Binding ELISA for the Different Fc Variants

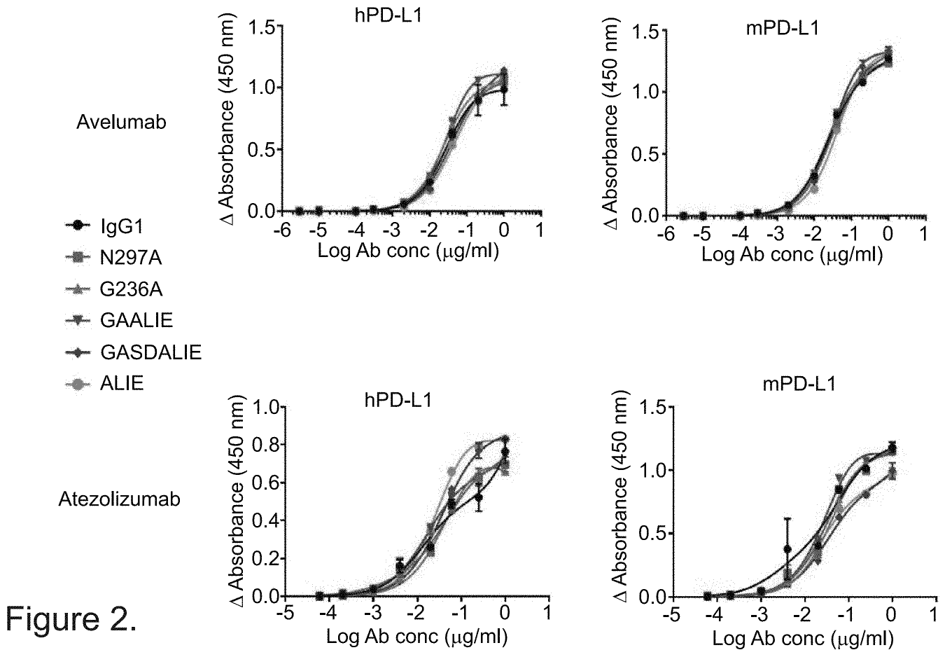

Avelumab and Atezolizumab variable domains (see FIG. 9) were synthesized. Standard cloning strategies were used to introduce the variable regions of these Abs into expression vectors that contain the IgG1 constant regions. Each anti-PD-L1 Ab clone was generated on the background of wild type IgG1, FcγR-binding null IgG1 (N297A), and a panel of Fc variants with defined FcγR-engagement characteristics with the different members of the human FcγR family (Table 2, below). As can be seen, “GASDALIE” (G236A/S239D/A330L/I332E; mainly enhanced FcRγIIIA binding, but also to FcγRIIA and FcγRIIB), A330L/I332E (selective enhancement of huFcγRIIIA binding), and G236A (selective enhancement of FcγRIIA binding). As shown in FIG. 2, Fc-engineering does not impair both mouse and human PD-L1 binding (FIG. 2).

TABLE 2
Binding affinities of the IgG Fc variants that will be used in the study to selected human FcγRs.

| | Inhibitory | | Activating | | | |
|---|---|---|---|---|---|---|
| | hFcγRIIB | | hFcγRIIA^{R131} | | hFcγRII^{F158} | |
| | K_{D} (μM) | Fold | K_{D} (μM) | Fold | K_{D} (μM) | Fold |
| IgG1-wild type | 3.01 | 1 | 1.16 | 1 | 6.7 | 1 |
| N297A | n.b | | n.b | | n.b | |
| G236A | 4.38 | 0.7 | 0.12 | 9.3 | 3.9 | 1.7 |
| G236A/S239D/A330L/I332E | 0.23 | 14 | 0.047 | 24.5 | 0.227 | 29.5 |
| A330L/I332E | 2.8 | 1.1 | 0.95 | 1.2 | | 5.4 |
| Afucosylated | 2.8 | 1.1 | 1.16 | 1 | 0.578 | 11.6 |

Binding constants were obtained by SPR analysis with immobilized FcγRs and soluble IgGs.
Bold = KD(IgG1)/KD(Fc variant);
n.b, no binding

### Example 4

### Abolishing huFc-FcγR Engagement from huIeG1 Subclass In Vivo Did not Affect Anti-Tumor Response in MC38 Tumor Model

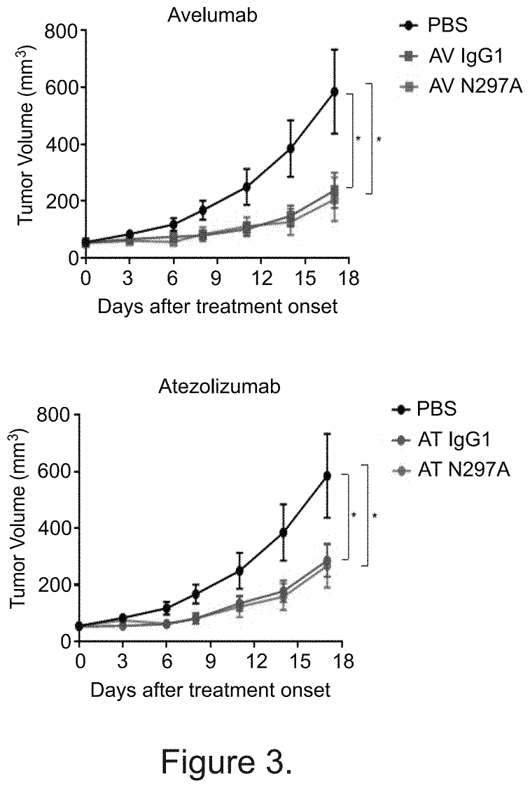

FcγR humanized mice with established MC38 tumors were treated with Avelumab or Atezolizumab. It was found that abolishing huFc-FcγR engagement from huIgG1 subclass in vivo did not affect the anti-tumor response of these Ab clones in MC38 tumor model (FIG. 3). Unlike the superior activity of the mouse IgG2a subclass, the human IgG1 subclass of anti-PD-L1 Abs did not result in improved antitumor activity compared to its Fc-silent (N297A) variant.

### Example 5

### FcγR Engagement of Atezolizumab Did not Alter Myeloid Cell Percentages in the TME

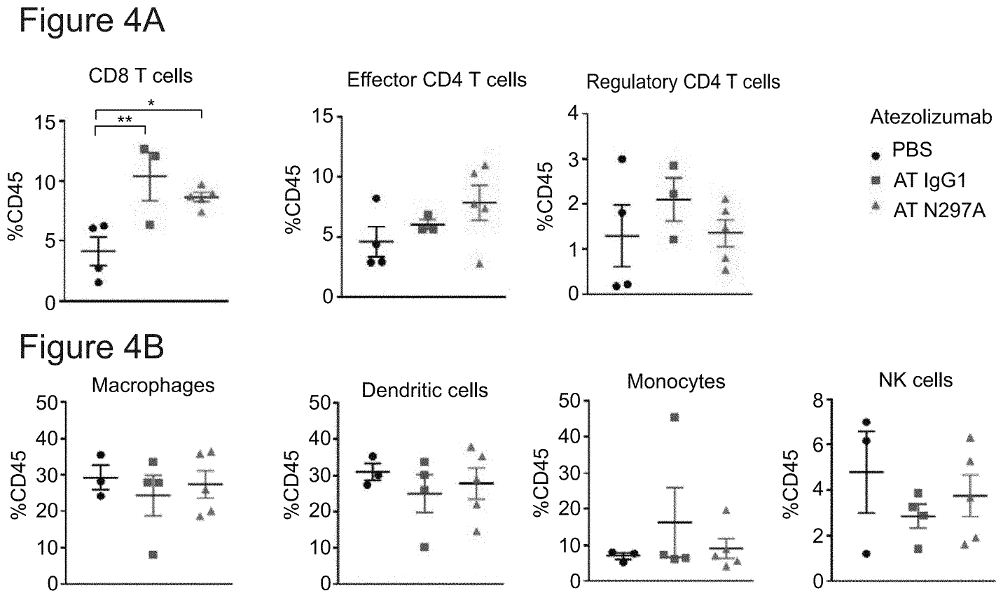

To better understand these results and to get a mechanistic insight into these FcγR-meditated pathways preliminary phenotyping of the TME composition was performed after anti-PD-L1 treatments. Similarly to the mouse, cytotoxic CD8 T cells were more abundant after anti-PD-L1 treatment and were not affected by Fc silencing (FIG. 4A). However, no modulation of myeloid populations was detected in the TME after treatment with neither the wild type nor the Fc-null versions (FIG. 4B).

The influence of Avelumab Fc glycan afucosylation was assessed on the Ab antitumor activity. It is shown that the absence of a fucose group from the Fc glycan resulted in specific increased affinity to FcγRIIIA (FIG. 6B) and improved antitumor activity (FIG. 7).

### Example 6

### Combined Targeting of huFcγRIIB and PD-L1 Increases the Therapeutic Effect of Avelumab in MC38 Tumor Model

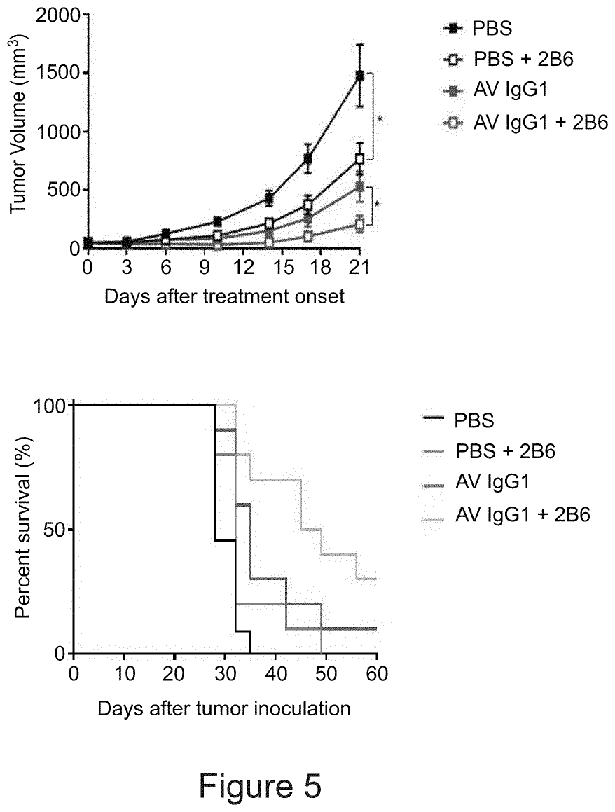

It has recently been discovered that FcγRIIB negatively regulates mAb-mediated immunotherapy and that targeting the inhibitory receptor can maximizes immunotherapy. FcγRIIB represents the sole FcγR with inhibitory activity that has the capacity to transduce intracellular signals that directly antagonize the immunostimulatory signals of activating FcγRs. Hence, it is possible that the therapeutic activity of human anti-PD-L1 mAbs is compromised by dominant engagement through the antibodies Fc domain with FcγRIIB over the activating FcγRs. Consistent with this hypothesis, combined targeting of huFcγRIIB and PD-L1 increased the therapeutic effect of Avelumab in MC38 tumor model. In vivo co-administration of huFcγRIIB blocker mAb (2B6) with Avelumab (IgG1) resulted in an increased anti-tumor response compared to treatment with Avelumab (IgG1) alone in MC38 tumor model (FIG. 5). As shown, administration of 2B6 alone resulted in an improved anti-tumor response compared to the control group.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1-2. (canceled)

3. A method of treating cancer, inflammatory disease or infectious disease in a subject in need thereof, the method comprising administering to the subject a therapeutically effective amount of each of a first binding moiety which specifically binds a human FcγRIIB and a second binding moiety which specifically binds a human PD-L1, wherein said second binding moiety which specifically binds said human PD-L1 is an antibody of a human IgG1 isotype, thereby treating cancer, inflammatory disease or infectious disease.

4. The method of claim 3, wherein said first binding moiety and said second binding moiety are in a co-formulation.

5. The method of claim 3, wherein said first binding moiety and said second binding moiety are in separate formulations.

6. The method of claim 3, wherein said first binding moiety and said second binding moiety compose a multispecific antibody.

7. The method of claim 3, wherein said first binding moiety and said second binding moiety compose a bispecific antibody.

8. The method of claim 3, wherein said first binding moiety which specifically binds said human FcγRIIB is an anti FcγRIIB antibody.

9. The method of claim 3, wherein said second binding moiety is Avelumab.

10. The method of claim 3, comprising a third binding moiety which binds a cancer antigen.

11. An anti PD-L1 antibody comprising an Fc region of a human IgG1 isotype having at least 95% identity to SEQ ID NO: 2, said antibody comprising complementary determining regions as set forth in SEQ ID NOs: 18-20 in a heavy chain with an N to C orientation and complementary determining regions as set forth in SEQ ID NOs: 21-23 in a light chain with an N to C orientation, said Fc region comprising at least one mutation and/or modification, which specifically enhances binding affinity of said Fc region to human FcγRIIA and/or FcγRIIIA as compared to wild type Fc region of said human IgG1.

12. The antibody of claim 11, wherein said at least one mutation is selected from the group consisting of S238D, S239D, I332E, A330L, S298A, E33A, L334A, G236A and L235V according to EU nomenclature.

13. The antibody of claim 12, wherein said at least one mutation comprises G236A/S239D/A330L/I332E.

14. (canceled)

15. The antibody of claim 11, wherein said modification is afucosylation.

16. A method of treating cancer, inflammatory disease or infectious disease in a subject in need thereof, the method comprising administering to the subject a therapeutically effective amount of the antibody of claim 11, thereby treating the cancer, inflammatory disease or infectious disease in the subject.

17. (canceled)

18. The method of claim 16 further comprising administering a therapeutically effective amount of a binding moiety which binds a cancer antigen.

19. (canceled)

20. The method of claim 16, wherein said cancer is a solid tumor cancer.

21. The method of claim 16, wherein said cancer is a non-solid tumor cancer.

22. An isolated polynucleotide comprising a nucleic acid sequence encoding the antibody of claim 11.

23. A nucleic acid construct comprising the nucleic acid sequence of claim 22 and a cis-acting regulatory sequence for driving expression of said nucleic acid sequence.

24. A cell comprising the nucleic acid construct of claim 23.

25. A method of producing an antibody, the method comprising:
(a) culturing the cells of claim 24 in a cell culture under conditions which allow expression of said antibody; and
(b) recovering the antibody from said cell culture.


SEQUENCE LISTING (34 sequences)

SEQ ID NO: 1 - protein, 329 aa, Homo sapiens
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 468 aa, Artificial sequence
  Avelumab wt heavy chain amino acid sequence
MRMLVLLYLLTALPGILSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 468 aa, Artificial sequence
  Avelumab N297 heavy chain amino acid sequence
MRMLVLLYLLTALPGILSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 468 aa, Artificial sequence
  Aveluman g236 heavy chain amino acid sequence
MRMLVLLYLLTALPGILSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 468 aa, Artificial sequence
  AVELUMAB GASDALIE amino acid sequence
MRMLVLLYLLTALPGILSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 468 aa, Artificial sequence
  Avelumab GAALIE amino acid sequence
MRMLVLLYLLTALPGILSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 468 aa, Artificial sequence
  Avelumab alie amino acid sequence
MRMLVLLYLLTALPGILSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 466 aa, Artificial sequence
  Atezo heavy amino acid sequence
MRMLVLLYLLTALPGILSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 466 aa, Artificial sequence
  Atezo n297 amino acid sequence
MRMLVLLYLLTALPGILSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 466 aa, Artificial sequence
  AtezoG236 amino acid sequence
MRMLVLLYLLTALPGILSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 466 aa, Artificial sequence
  Atezo GASDALIE amino acid sequence
MRMLVLLYLLTALPGILSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 466 aa, Artificial sequence
  Atezo GAALIE amino acid sequence
MRMLVLLYLLTALPGILSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 466 aa, Artificial sequence
  Atezo ALIE amino acid sequence
MRMLVLLYLLTALPGILSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 470 aa, Artificial sequence
  2B6 297 HEAVY CHAIN amino acid sequence
MGWSCIILFLVATATGVHSQVQLQQPVTELVRPGASVMLSCKASDYPFTNYWIHWVKQRPGQGLEWIGVIDPSDTYPNYNKKFKGKATLTVVVSSSTAYMQLSSLTSDDSAVYYCARNGDSDYYSGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 233 aa, Artificial sequence
  2B6 297 LIGHT chain amino acid sequence
MGWSCIILFLVATATGVHSDILLTQSPAILSVSPGERVSFSCRTSQSIGTNIHWYQQRTNGFPRLLIKNVSESISGIPSRFSGSGSGTDFILSINSVESEDIADYYCQQSNTWPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 463 aa, Artificial sequence
  HEAVY CHAIN D265 2B6 amino acid sequence
  misc_feature (229)..(229): Xaa can be any naturally occurring amino acid
MRMLVLLYLLTALPGILSQVQLQQPVTELVRPGASVMLSCKASDYPFTNYWIHWVKQRPGQGLEWIGVIDPSDTYPNYNKKFKGKATLTVVVSSSTAYMQLSSLTSDDSAVYYCARNGDSDYYSGMDYWGQGTSVTVSSATTKGPSVYPLAPGSAAQTNSMVTLGCLVKGYFPEPVTVTWNSGSLSSGVHTFPAVLQSDLYTLSSSVTVPSSTWPSQTVTCNVAHPASXTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 17 - protein, 234 aa, Artificial sequence
  LIGHT CHAIN D265 286 amino acid sequence
MRCSLQFLGLLVLWIPGLNGDILLTQSPAILSVSPGERVSFSCRTSQSIGTNIHWYQQRTNGFPRLLIKNVSESISGIPSRFSGSGSGTDFILSINSVESEDIADYYCQQSNTWPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 18 - protein, 5 aa, Artificial sequence
  Complementarity-determining region (CDR) amino acid sequence
SYIMM

SEQ ID NO: 19 - protein, 17 aa, Artificial sequence
  Complementarity-determining region (CDR) amino acid sequence
SIYPSGGITFYADTVKG

SEQ ID NO: 20 - protein, 11 aa, Artificial sequence
  Complementarity-determining region (CDR) amino acid sequence
IKLGTVTTVDY

SEQ ID NO: 21 - protein, 14 aa, Artificial sequence
  Complementarity-determining region (CDR) amino acid sequence
TGTSSDVGGYNYVS

SEQ ID NO: 22 - protein, 7 aa, Artificial sequence
  Complementarity-determining region (CDR) amino acid sequence
DVSNRPS

SEQ ID NO: 23 - protein, 10 aa, Artificial sequence
  Complementarity-determining region (CDR) amino acid sequence
SSYTSSSTRV

SEQ ID NO: 24 - protein, 8 aa, Artificial sequence
  Complementarity-determining region (CDR) amino acid sequence
GFTFSDSW

SEQ ID NO: 25 - protein, 8 aa, Artificial sequence
  Complementarity-determining region (CDR) amino acid sequence
ISPYGGST

SEQ ID NO: 26 - protein, 11 aa, Artificial sequence
  Complementarity-determining region (CDR) amino acid sequence
ARRHWPGGFDY

SEQ ID NO: 27 - protein, 6 aa, Artificial sequence
  Complementarity-determining region (CDR) amino acid sequence
QDVSTA

SEQ ID NO: 28 - protein, 3 aa, Artificial sequence
  Complementarity-determining region (CDR) amino acid sequence
SAS

SEQ ID NO: 29 - protein, 9 aa, Artificial sequence
  Complementarity-determining region (CDR) amino acid sequence
QQYLYHPAT

SEQ ID NO: 30 - protein, 470 aa, Homo sapiens
MGWSCIILFLVATATGVHSQVQLQQPVTELVRPGASVMLSCKASDYPFTNYWIHWVKQRPGQGLEWIGVIDPSDTYPNYNKKFKGKATLTVVVSSSTAYMQLSSLTSDDSAVYYCARNGDSDYYSGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 233 aa, Artificial sequence; identical to SEQ ID NO: 15
  2B6 WT LIGHT CHAIN amino acid sequence

SEQ ID NO: 32 - protein, 235 aa, Artificial sequence
  LIGHT CHAIN  AVE amino acid sequence
MGWSCIILFLVATATGVHSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 33 - protein, 233 aa, Artificial sequence
  LIGHT CHAIN ATEZO amino acid sequence
MGWSCIILFLVATATGVHSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 463 aa, Artificial sequence
  2B6 mouse WT HEAVY CHAIN amino acid sequence
MRMLVLLYLLTALPGILSQVQLQQPVTELVRPGASVMLSCKASDYPFTNYWIHWVKQRPGQGLEWIGVIDPSDTYPNYNKKFKGKATLTVVVSSSTAYMQLSSLTSDDSAVYYCARNGDSDYYSGMDYWGQGTSVTVSSATTKGPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK